(19) United States
(12) Patent Application Publication
(10) Pub. No.: US 2026/0364281 A1
(43) Pub. Date: Nov. 19, 2026

(54) INHALATION DEVICE ROTARY ACTUATOR

(71) Applicant: invoX Belgium NV, Diepenbeek (BE)

(72) Inventors: Juergen Rawert, Koeln (DE); Steven Dudley, Brighton (GB); Steven Madden, Hamburg (DE)

(21) Appl. No.: 19/580,643

(22) PCT Filed: Oct. 24, 2024
(86) PCT No.: PCT/EP2024/080004

§ 371 Date: Apr. 22, 2026

(30) Foreign Application Priority Data
Oct. 25, 2023 (EP) 23205752.1

(51) Int. Cl. A61M 15/00

(52) U.S. Cl. CPC A61M 15/0006; A61M 15/009

(57) ABSTRACT

An inhalation device (100) is disclosed that can include a first housing (300) and a connecting member (200) at least partially disposed within the first housing (300) of the inhaler device (100). The inhalation device (100) can also include a button (250) secured to the first housing (300) and further engaged with the connecting member (200), and a biasing member (106) secured to one end of the connecting member (200), such that the connecting member (200) is adapted to at least partially reciprocate within the first housing (300) in order for the inhaler device (100) to dispense a liquid. In addition, the connecting member (200) may include a first cam member (210) and a second cam member (220) on its exterior region. Further, the connecting member (200) may include a first follower member (230) and a second follower member (240) in its interior region.

### FIELD OF THE INVENTION

The present disclosure described herein relates to inhalation devices such as portable inhalation devices.

### BACKGROUND OF THE INVENTION

This section is intended to introduce the reader to aspects of art that may be related to various aspects of the present disclosure described herein, which are described and/or claimed below. This discussion is believed to be helpful in providing the reader with background information to facilitate a better understanding of the various aspects of the present disclosure described herein. Accordingly, it should be understood that these statements are to be read in this light, and not as admissions of prior art.

Nebulizers, inhalers, or other aerosol generators for liquids have been known from the art since a long time ago. Amongst others, such devices are used in medical science and therapy. There, they serve as inhalation devices for the application of active ingredients in the form of aerosols, (i.e., small, atomized liquid droplets embedded in a gas). Some general components of such inhalation devices can include a reservoir in which the liquid that is to be aerosolized is contained therein, a pumping unit for generation of a pressure being sufficiently high for nebulizing, as well as an atomizing device in the form of a nozzle, among others. A pumping unit can generally be a component capable of moving or compressing a fluid material and that can include at least one pumping chamber, and optionally further include auxiliary components as well. The pumping unit can draw liquid in a discrete amount from the reservoir and feed it to the nozzle for dispensing. However, many of the conventional inhalation devices include multiple parts that contribute to more complex manufacturing and higher costs for such devices.

For example, US 2022/0323696 (Stuart) discloses a portable inhaler having a rotary actuation mechanism, a follower component, a rotary loading device, and a button. The rotary loading device includes a cam component engaged with a cage component, to allow a predetermined limited rotational relative movement therebetween. However, the portable inhaler of Stuart requires multiple independent parts which subsequently increases the cost of manufacturing and the complexity of assembly. Further, these additional components may further expose the inhaler to jamming and inoperability as the inhaler is repeatedly actuated.

Hence, what is needed is an inhalation device that allows a user to prime the inhalation device via simple rotational movement and to conveniently, reliably, efficiently, and accurately dispense a discrete or pre-defined amount of liquid or medication, and that further includes minimum parts for its manufacture.

### SUMMARY OF THE INVENTION

In one aspect of the disclosure described herein, an inhalation device such as a portable inhaler is disclosed. The inhalation device can include a first housing, a connecting member at least partially disposed within the first housing of the inhaler device. The inhalation device can also include a button secured to the first housing and further engaged with the connecting member, and a biasing member secured to one end of the connecting member, such that the connecting member is adapted to at least partially reciprocate within the first housing in order for the inhaler device to dispense a liquid.

In addition, the connecting member may include a first cam member and a second cam member on its exterior region.

Further, the connecting member may include a first follower member and a second follower member in its interior region.

Also, the first housing may further include a third cam member and a fourth cam member, wherein the third cam member is adapted to engage the first follower member and the fourth cam member is adapted to engage the second follower member.

Moreover, the button may include a third follower member, wherein the third follower member is adapted to engage either the first cam member or the second cam member.

In addition, the inhalation device may further include a second housing including a channel, and the connecting member may further include a tab member, wherein the tab member is adapted to at least partially rotate within the channel.

Also, the tab member may be adapted to rotate up to about 7-degrees within the channel of the second housing.

Further, the inhalation device may include a sliding member, wherein the sliding member is adapted to slide within the channel of the second housing.

In addition, the third cam member and fourth cam member may each be in a helical or spiral configuration.

Moreover, the first follower member and the second follower member may each include a flat surface region followed by a sloped surface region.

In addition, the first cam member and the second cam member may each be disposed at about 180-degrees relative to each other.

Also, the first follower member and the second follower member may each be disposed at about 180-degrees relative to each other.

Further, the inhalation device may further comprise a riser pipe at least partially disposed through the center of the connecting member.

Also, the connecting member or reciprocating member may be adapted to reciprocate relative to the riser pipe.

In addition, the riser pipe may be fixed relative to the first housing, and the connector reciprocates and moves as a pumping unit relative to the riser pipe for dispensing the liquid.

Moreover, the biasing member may be a compression spring secured to one end of the connecting member.

In another aspect of the present disclosure described herein, an inhalation device such as a portable inhaler is disclosed having a pumping unit including a connecting member; a button engaged with the connecting member; and a biasing member secured to one end of the connecting member, such that the connecting member is adapted to reciprocate within a first housing of the inhalation device to dispense a liquid.

In addition, the connecting member may further include a first cam member and a second cam member on its exterior region. In some cases, the first cam member and second cam member may each be comprised of sloping surfaces.

Further, the connecting member may include a first follower member and a second follower member in its interior region.

Also, the inhalation device may further include a third cam member and a fourth cam member, wherein the third cam member is adapted to engage the first follower member and the fourth cam member is adapted to engage the second follower member. In some cases, the third cam member and fourth cam member may each be comprised of sloping surfaces.

Moreover, the button may include a third follower member, wherein the third follower member is adapted to engage either the first cam member or the second cam member.

In addition, the inhalation device may further include a second housing including a channel, and the connecting member further includes a tab member, wherein the tab member is adapted to at least partially rotate within the channel.

Also, the tab member may be adapted to rotate up to about 7-degrees within the channel of the second housing.

Further, the inhalation device may include a sliding member, wherein the sliding member may be adapted to slide within the channel of the second housing.

In addition, the third cam member and fourth cam member may each include a helical or spiral configuration.

Also, the first cam member and the second cam member may each be disposed at 180-degrees relative to each other.

Moreover, the first follower member and the second follower member may each be disposed at 180-degrees relative to each other.

Further, a riser pipe may be at least partially disposed through a center of the connecting member.

In addition, the connecting member may be adapted to reciprocate relative to the riser pipe.

Also, the riser pipe may be fixed relative to the first housing and the connector reciprocates and moves as a pumping unit relative to the riser pipe for dispensing the liquid.

Moreover, the biasing member may be a compression spring secured to one end of the connecting member.

In another aspect of the present disclosure described herein, an inhalation device such as a portable inhaler is disclosed as having a rotary actuation mechanism. Here, the inhalation device can include a first housing, a connecting member, and a button secured to the first housing and further engaged with an outer wall of the connecting member. The connecting member may include a plurality of cam or follower member surfaces within its interior region and on its exterior region. In addition, a biasing member (e.g., a compression spring) can be secured to one end of the connecting member, such that the connecting member is adapted to reciprocate within the first housing in order for the inhalation device to dispense a liquid. In some embodiments, the liquid is a single or discrete dose of the liquid, for example, a medicament.

In another aspect of the present disclosure described herein, an inhalation device such as a portable inhaler is disclosed having a first housing including a first cam member; a connecting member at least partially disposed within the first housing, wherein an interior of the connecting member further comprises a first protrusion engaged with the first cam member of the first housing, and wherein an exterior of the connecting member further comprises a second cam member; and a button secured to the first housing, wherein the button further comprises a second protrusion engaged with the second cam member of the connecting member.

The above summary is not intended to describe each and every disclosed embodiment or every implementation of the disclosure. The description that follows more particularly exemplifies the various illustrative embodiments.

### BRIEF DESCRIPTION OF THE DRAWINGS

The following description should be read with reference to the drawings, in which like elements in different drawings are numbered in like fashion. The drawings, which are not necessarily to scale, depict selected embodiments and are not intended to limit the scope of the disclosure. The disclosure may be more completely understood in consideration of the following detailed description of various embodiments in connection with the accompanying drawings, in which:

FIG. 1A illustrates a perspective view of the inhalation device of the present disclosure described herein, according to one or more non-limiting exemplary embodiments.

FIG. 1B illustrates another perspective view of the inhalation device of FIG. 1A.

FIG. 2 illustrates an exploded view of the inhalation device of the present disclosure described herein, according to one or more non-limiting exemplary embodiments.

FIG. 3 illustrates a partial side view of the inhalation device of the present disclosure described herein, according to one or more non-limiting exemplary embodiments.

FIG. 4A illustrates a partial cross-sectional side view of the inhalation device of the present disclosure described herein, according to one or more non-limiting exemplary embodiments.

FIG. 4B illustrates a partial cross-sectional front view of the inhalation device of the present disclosure described herein, according to one or more non-limiting exemplary embodiments.

FIG. 5 illustrates a partial perspective top view of the inhalation device of the present disclosure described herein, according to one or more non-limiting exemplary embodiments.

FIG. 6 illustrates a top view of the inhalation device of the present disclosure described herein, according to one or more non-limiting exemplary embodiments.

FIG. 7A illustrates a partial perspective view of an interior of the first housing of the inhalation device of the present disclosure described herein, according to one or more non-limiting exemplary embodiments.

FIG. 7B illustrates another partial perspective view of the interior of the first housing of the inhalation device of the present disclosure described herein, according to one or more non-limiting exemplary embodiments.

FIG. 8A illustrates a partial perspective view of the inhalation device of the present disclosure described herein, according to one or more non-limiting exemplary embodiments.

FIG. 8B illustrates a partial close-up, side view of a blocking member of the inhalation device of the present disclosure described herein, according to one or more non-limiting exemplary embodiments.

FIG. 8C illustrates a partial close-up perspective view of the blocking member of the inhalation device of the present disclosure described herein.

FIGS. 9A-9D illustrate top views of the inhalation device of the present disclosure described herein, further illustrating a method of operation according to one or more non-limiting exemplary embodiments.

FIGS. 10A-10D illustrate partial perspective views of the inhalation device of the present disclosure described herein, further illustrating a method of operation according to one or more non-limiting exemplary embodiments.

FIGS. 11A-11D illustrate partial simplified perspective side views of the inhalation device of the present disclosure described herein, further illustrating a method of operation, according to one or more non-limiting exemplary embodiments.

### DETAILED DESCRIPTION OF THE INVENTION

In the Brief Summary of the present disclosure above and in the Detailed Description of the disclosure described herein, and the claims below, and in the accompanying drawings, reference is made to particular features (including method steps) of the disclosure described herein. It is to be understood that the disclosure of the disclosure described herein in this specification includes all possible combinations of such particular features. For example, where a particular feature is disclosed in the context of a particular aspect or embodiment of the disclosure described herein, or a particular claim, that feature can also be used, to the extent possible, in combination with and/or in the context of other particular aspects and embodiments of the disclosure described herein, and in the disclosure described herein generally.

The embodiments set forth below represent the necessary information to enable those skilled in the art to practice the disclosure described herein and illustrate the best mode of practicing the disclosure described herein. In addition, the disclosure described herein does not require that all the advantageous features and all the advantages need to be incorporated into every embodiment of the disclosure described herein.

As used herein, the terms “connector” or “connecting member” may be used interchangeably, and can be any type of body (e.g., hollow body, solid body, or partially hollow body), capsule, holder, connector, connecting member, reciprocating member, or translating member, among others.

As used herein, a “cam” can be any type of driving member and a “follower” can be any member driven by the cam as is ordinarily understood in the technical field concerned. Here, the shape or profile of the cam influences the motion of the follower. Further, as used herein, a cam and a follower may be used interchangeably.

As used herein, “about”, “approximately”, “substantially” and the like in connection with an attribute or value include the exact attribute or the precise value, as well as any attribute or value typically considered to fall within a normal range or variability accepted in the technical field concerned.

The disclosure provides an inhalation device (100) such as a portable inhaler having a first housing (300), a connecting member (200) having a first end at least partially disposed within the first housing (300) of the inhaler (100). The inhaler (100) can further include a button (250) secured to the first housing (300). Here, the button (250) may be adapted to engage the connecting member (200). In some embodiments, the button (250) can include a third follower member (252) (also referred to herein as a “protruding member” or “protrusion”) about its middle region, wherein the third follower member (252) is adapted to engage the connecting member (200). In addition, a biasing member (106) can be secured to one end of the connecting member (200), such that the connecting member (200) is adapted to linearly reciprocate within the first housing (300) in order for the inhaler (100) to dispense a liquid. In some embodiments, the inhaler device (100) dispenses a single dose of a liquid, for example, a medicament.

In a first aspect, an inhalation device such as a portable inhaler is disclosed having a first housing (300), a connecting member (200) at least partially disposed within the first housing (300) of the inhaler. The connecting member (200) can include a first cam member (210) and a second cam member (220) on its exterior region, and a first follower member (230) and a second follower member (240) in its interior region. The inhaler (100) can further include a button (250) secured to the first housing (300) and further engaged with the first cam member (210) or the second cam member (220) of the connecting member (200). In addition, a biasing member (106) can be secured to one end of the connecting member (200), such that the connecting member (200) is adapted to linearly reciprocate within the first housing (300) in order for the inhaler (100) to dispense a liquid. In some embodiments, the inhaler device (100) dispenses a single dose of a liquid, for example, a medicament. Further, the first housing (300) may include a third cam member (330) and a fourth cam member (340), wherein the third cam member (330) is adapted to engage the first follower member (230) and the fourth cam member (340) is adapted to engage the second follower member (240). In certain embodiments, the first (210), second (220), third (330) and fourth (340) cam members may each be comprised of sloping surfaces.

In other embodiments of the first aspect, the button (250) of the inhalation device (100) may include a third follower member (252), wherein the third follower member (252) is adapted to engage either the first cam member (210) or the second cam member (220). In addition, the inhalation device (100) may further include a second housing (110) including a channel (110B), and the connecting member (200) may further include a tab member (202), wherein the tab member (202) can be adapted to at least partially rotate within the channel (110B). In specific embodiments, the tab member (202) of the connecting member (200) may be a block member. In some embodiments, the channel (110B) may be a groove or a cut-out in the second housing (110). In addition, the tab member (202) can be adapted to rotate up to about 7-degrees in any direction within the channel (110B) of the second housing (110).

In other embodiments of the first aspect, the inhalation device (100) may further include a sliding member (114), wherein the sliding member (114) is adapted to slide within the channel (110B) of the second housing (110). In addition, the third cam member (330) and fourth cam member (340) of the first housing (300) can each be disposed in a helical or spiral configuration. Here, the third and fourth cam members (330, 340) may have any type of slope or grade. In addition, the first follower member (230) and the second follower member (240) may each include a flat surface region followed by a sloped surface region.

In other embodiments of the first aspect, the first cam member (210) and the second cam member (220) may each be disposed at about 180-degrees relative to each other. Further, the first follower member (230) and the second follower member (240) may each be disposed at about 180-degrees relative to each other. Further, a riser pipe (112) may be at least partially disposed through a center of the connecting member (200). In addition, the connecting member (200) may be adapted to reciprocate relative to the riser pipe (112). Also, the riser pipe (112) may be fixed relative to the first housing (300) and the connector (200) reciprocates and moves as a pumping unit relative to the riser pipe (112) for dispensing the liquid.

In other embodiments of the first aspect, the biasing member (106) is a compression spring that can be secured to one end of the connecting member (200), such that the connecting member (200) is adapted to reciprocate within the first housing (300) in order for the inhalation device (100) to dispense a liquid. In some embodiments, the liquid is a single or discrete dose of the liquid, for example, a medicament.

In a second aspect, an inhalation device (100) such as a portable inhaler is disclosed having a pumping unit comprised of a connecting member (200), a button (250) engaged with the connecting member (200), and a biasing member (106) secured to one end of the connecting member (200), such that the connecting member (200) is adapted to reciprocate within the first housing (300) to dispense a liquid. In some embodiments, the connecting member (200) can include a first cam member (210) and a second cam member (220) on its exterior region, and a first follower member (230) and a second follower member (240) in its interior region. The button (250) may be secured to the first housing (300) and further engaged with the first cam member (210) or the second cam member (220) of the connecting member (200).

Further, the first housing (300) may include a third cam member (330) and a fourth cam member (340), wherein the third cam member (330) is adapted to engage the first follower member (230) and the fourth cam member (340) is adapted to engage the second follower member (240). In certain embodiments, the first (210), second (220), third (330), and fourth cam members (340) may each be comprised of sloping surfaces.

In other embodiments of the second aspect, the button (250) of the inhalation device (100) may include a third follower member (252), wherein the third follower member (252) is adapted to engage either the first cam member (210) or the second cam member (220). In addition, the inhalation device (100) may further include a second housing (110) including a channel (110B), and the connecting member (200) may further include a tab member (202), wherein the tab member (202) can be adapted to at least partially rotate within the channel (110B). In addition, the tab member (202) can be adapted to rotate up to about 7-degrees in any direction within the channel (110B) of the second housing (110).

In other embodiments of the second aspect, the inhalation device (100) may further include a sliding member (114), wherein the sliding member (114) is adapted to slide within the channel (110B) of the second housing (110). In addition, the third cam member (330) and fourth cam member (340) of the first housing (300) can each be in a helical or spiral configuration. Here, the third and fourth cam members (330, 340) may have any type of slope or grade. In addition, the first follower member (230) and the second follower member (240) may each include a flat surface region followed by a sloped surface region.

In other embodiments of the second aspect, the first cam member (210) and the second cam member (220) may each be disposed at about 180-degrees relative to each other. Further, the first follower member (230) and the second follower member (240) may each be disposed at about 180-degrees relative to each other. Further, a riser pipe (112) may be at least partially disposed through a center of the connecting member (200). In addition, the connecting member (200) may be adapted to reciprocate relative to the riser pipe (112). Also, the riser pipe (112) may be fixed relative to the first housing (300) and the connector (200) reciprocates and moves as a pumping unit relative to the riser pipe (112) for dispensing the liquid.

In other embodiments of the second aspect, the biasing member (106), for example, is a compression spring that can be secured to one end of the connecting member (200), such that the connecting member (200) is adapted to reciprocate within the first housing (300) in order for the inhalation device (100) to dispense a liquid. In some embodiments, the liquid is a single or discrete dose of the liquid, for example, a medicament.

In a third aspect, an inhalation device (100) such as a portable inhaler is disclosed having a rotary actuation mechanism. Here, the inhalation device (100) can include a first housing (300), a connecting member (200), and a button secured (250) to the first housing (300) and further engaged with an outer wall of the connecting member (200). The connecting member (200) may include a plurality of cam and/or follower member surfaces within its interior region and on its exterior region. In addition, a biasing member (106) (e.g., a compression spring) can be secured to one end of the connecting member (200), such that the connecting member (200) is adapted to reciprocate within the first housing (300) in order for the inhalation device to dispense a liquid. In some embodiments, the liquid is a single or discrete dose of the liquid, for example, a medicament.

In a fourth aspect, an inhalation device (100) such as a portable inhaler is disclosed having a first housing (300) comprising a first cam member; a connecting member (200) at least partially disposed within the first housing (300), wherein an interior of the connecting member (200) further includes a first protrusion engaged with the first cam member of the first housing (300), and wherein an exterior of the connecting member (200) further includes a second cam member; and a button (250) secured to the first housing (300), wherein the button (250) further includes a second protrusion (252) engaged with the second cam member of the connecting member (200). In addition, a biasing member (106) (e.g., a compression spring) can be secured to one end of the connecting member (200), such that the connecting member (200) is adapted to reciprocate within the first housing (300) in order for the inhalation device (100) to dispense a liquid. In some embodiments, the liquid is a single or discrete dose of the liquid, for example, a medicament.

### DETAILED DESCRIPTION OF THE DRAWINGS

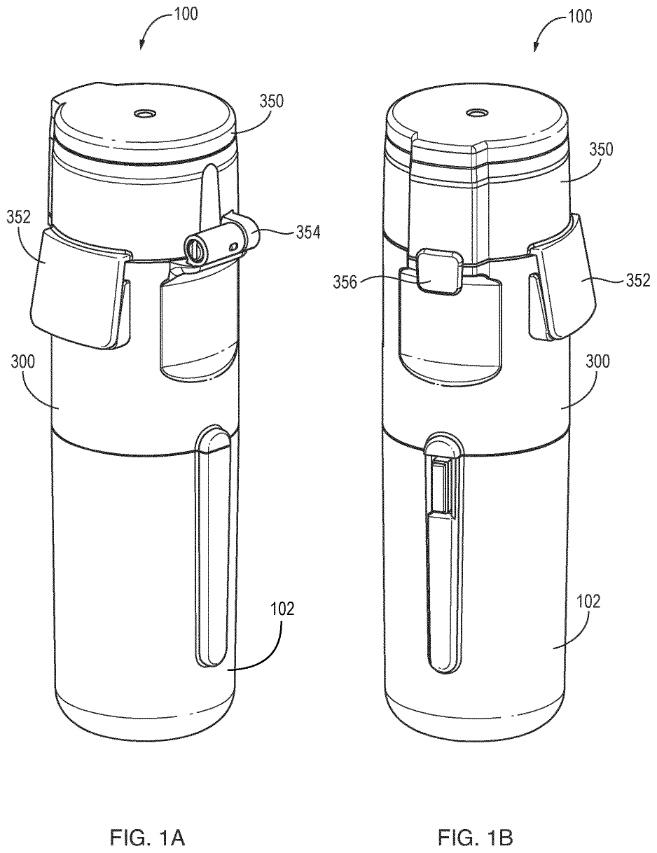

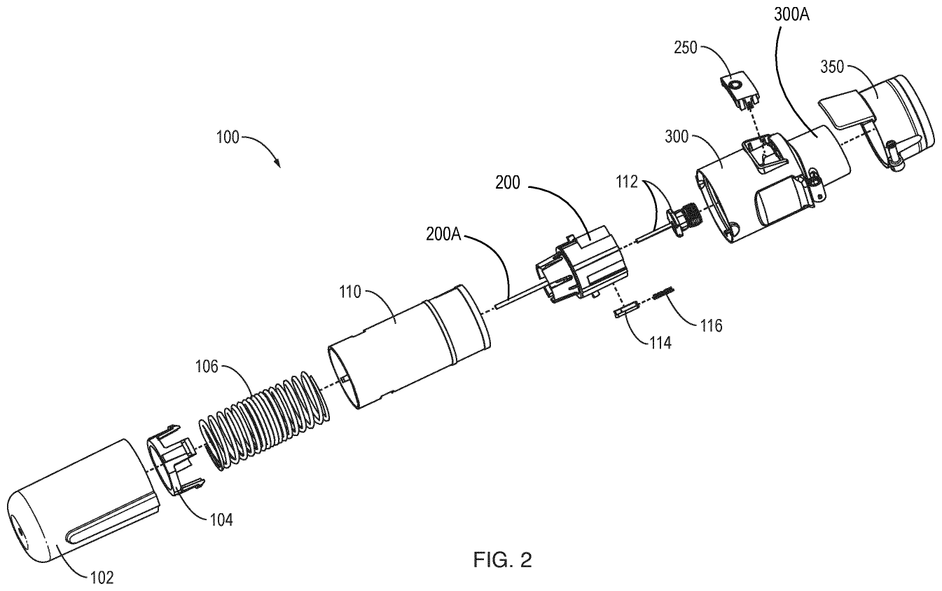

FIGS. 1A-11D illustrate one or more non-limiting embodiments of the inhaler device (100) of the present disclosure described herein for dispensing a single, discrete, or pre-defined dosage of atomized liquid or medicament. Referring to FIGS. 1A-1B, device (100) can include a first housing (300) (also referred to herein as an “upper body”), a second housing (110) (also referred to herein as an “intermediate body”, FIG. 2) and a third housing (102) (also referred to herein as a “lower body”). The first housing (300) and third housing (102) may be external housings while the second housing (110) may be an intermediate housing that is housed inside the first (300) and third (102) housings. The third housing (102) may be secured to the first housing (300). First housing (300) can further include a removable cap (350) secured thereto, wherein cap (350) can pivot about hinge (354) in order to access a mouthpiece component (300A) (FIG. 2). In particular, cap (350) can include a locking and release member button (356) that allows cap (350) to release and open (via rotation about hinge (354)) in order to expose the mouthpiece component (300A), and further allow cap (350) to be closed and secured via button (356), thereby covering and enclosing mouthpiece component (300A). Cap (350) can include a cover or tab member (352) that can cover button (250) of the inhaler device in order to prevent inadvertent engagement of button (250).

FIG. 2 illustrates an exploded view of the inhaler device (100) of the present disclosure described herein. Here, device (100) is shown with third housing (102) enclosing therein a spring support member (104) which receives therein one end of biasing member (106). In specific embodiments, the biasing member (106) may be, for example, a spring or a compression spring and may be referred to as such. In addition, an internal or intermediate body or second housing (110) can be disposed over biasing member (106) and secured to and engaged with one or more tabs on support member (104), wherein biasing member (106) is disposed within second housing (110). In addition, connecting member (200) is further partially received within second housing (110) such that the connector (200) engages an opposing end of biasing member (106). In operation, connector (200) is adapted to move linearly within second housing (110) via biasing member (106) (e.g., a compression spring), which will later be discussed in more detail. Connector (200) can further operate as part of the pumping chamber, pumping unit, or dosing chamber of device (100), wherein the pumping chamber can also include riser pipe and holder (112). Still referring to FIG. 2, riser pipe and holder (112) can be axially aligned with the center of connector (200) and secured thereto, such that the riser pipe (112) can be in fluid communication with piping (200A) that is in further fluid communication with a reservoir (not shown) for dispensing a liquid/medicament. In one embodiment, riser pipe (112) is fixed relative to first housing (300), and connector (200) and piping (200A) reciprocate and move (as a pumping unit) relative to the riser pipe (112) for dispensing the liquid from the reservoir. In addition, FIG. 2 illustrates mouthpiece component (300A) which can be housed by cap (350). Also, button (250) is shown which can engage connector (200), which will later be discussed in more detail. Further, a sliding member (114) and slider spring (116) are shown that are secured to connector (200), which will later be discussed in more detail. Here, button (250) is also secured to second housing (110) and first housing (300).

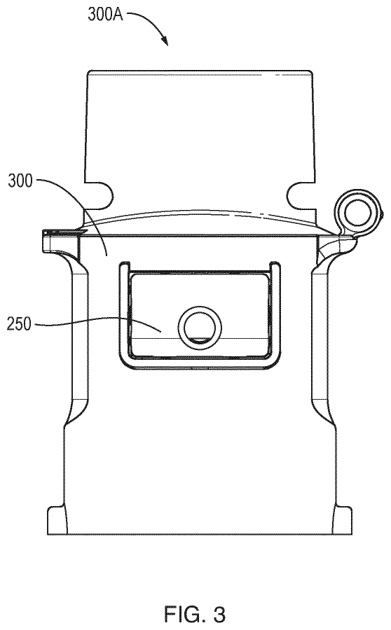

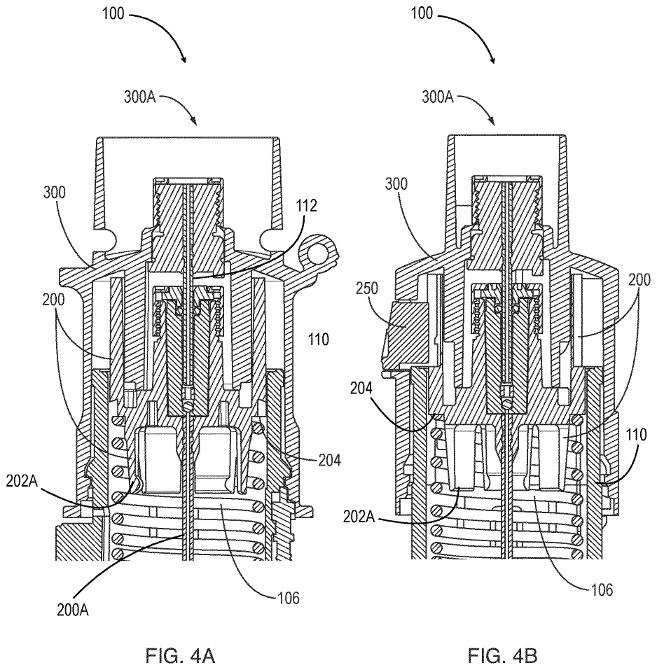

FIG. 3 illustrates a front view of first housing (300) of the device (100). FIG. 4A illustrates a partial cross-sectional side view of the upper region of device (100) and FIG. 4B illustrates a partial cross-sectional front view of the upper region of device (100). Referring to FIGS. 4A-4B, device (100) is shown with one end of spring (106) engaging connector (200). Specifically, connector (200) can include a cut-out or ledge (204) around its perimeter wherein spring (106) can be seated therein or thereon. In addition, connector (200) can include a plurality of protrusions, prongs, or tabs (202A) disposed within the interior space of spring (106).

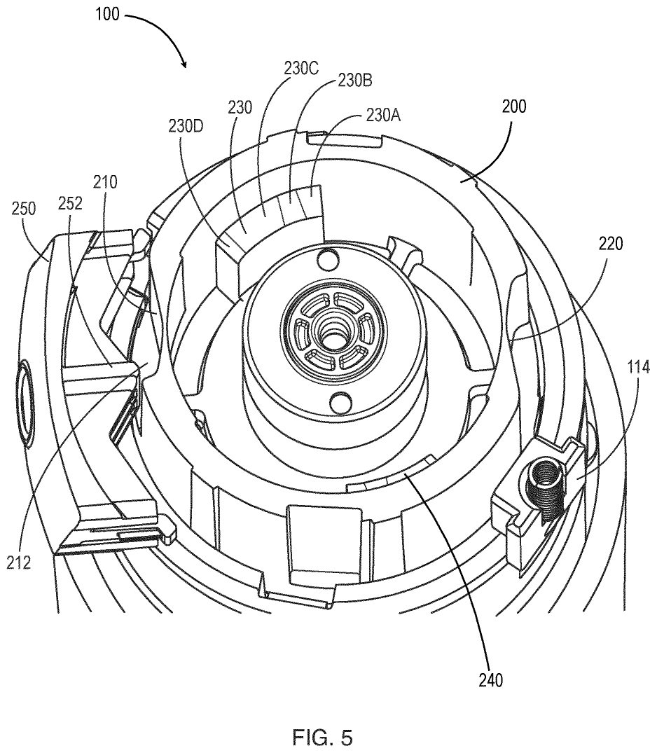

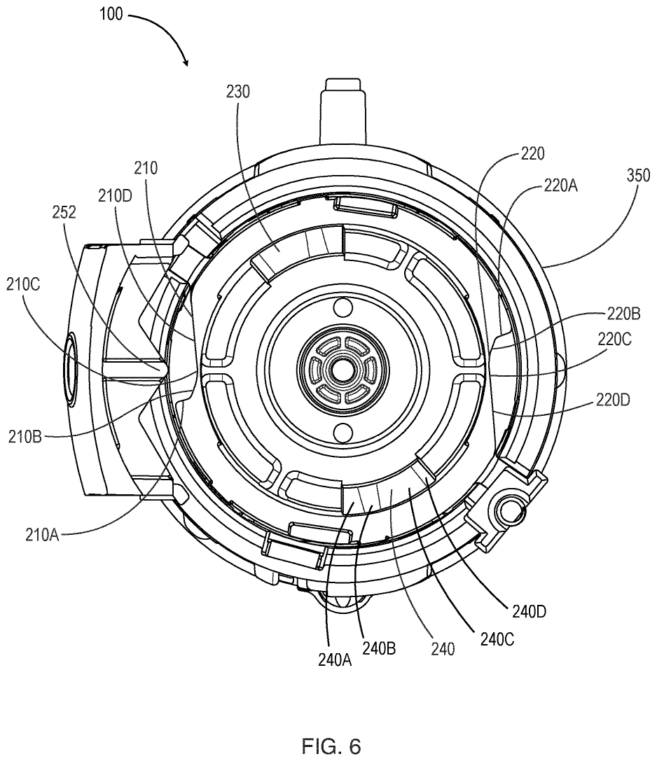

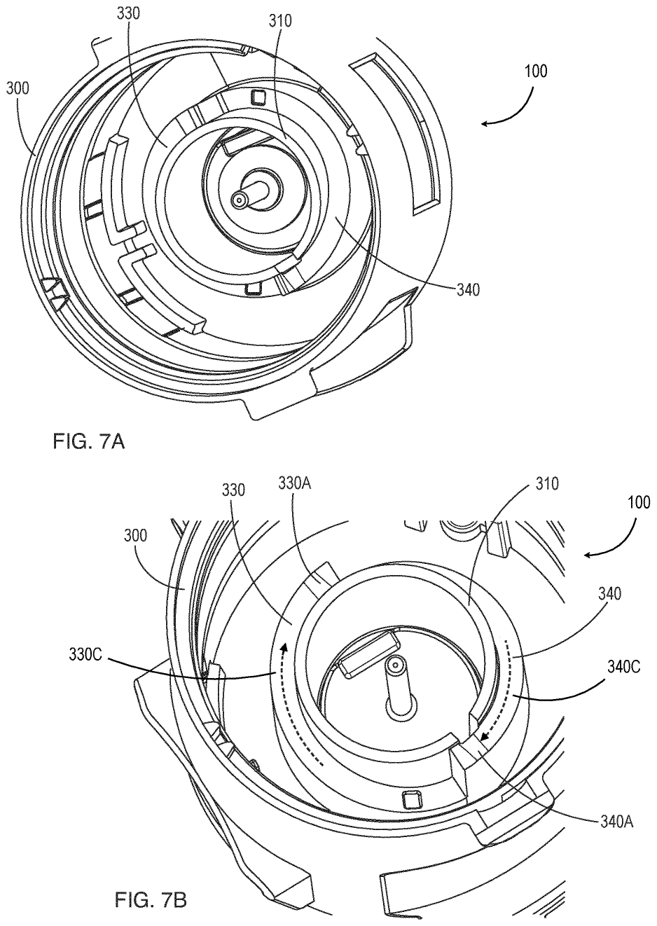

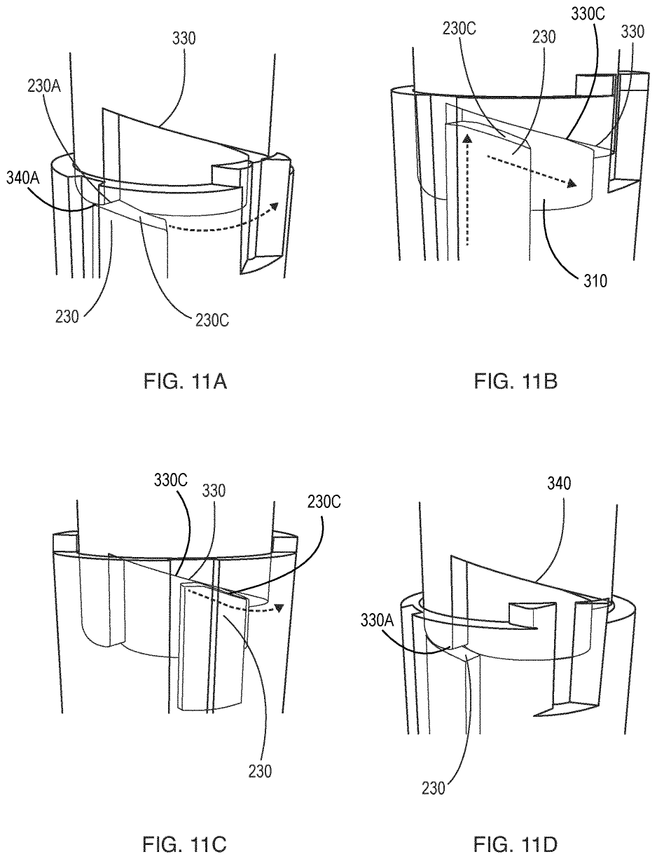

FIGS. 5-6 illustrate various views of the connector (200) and button (250) of the inhaler device (100). As shown, connector (200) can include a radial cam or follower member (230) comprised of a projection having a declining ramp that starts at a high top flat region, ledge, or plateau (230A) followed by a first lower sloping region (230B) followed by a second lower sloping region (230C) and further followed by a third lower sloping region (230D), wherein third lower sloping region (230D) is lower in height than the high top flat region, ledge, or plateau (230A). Specifically, high top flat region (230A) can be comprised of a flat smooth surface that engages either of ledge or plateau regions (330A) or (340A) of opposing third and fourth cam components (330) or (340), respectively, such as shown in FIGS. 7A-7B and 11A. Further, second lower sloping region (230C) can abut, translate, engage, and slide along either of opposing downward sloping regions (330C) or (340C) of opposing third and fourth cam components (330) or (340), respectively, such as shown in FIGS. 7A-7B and 11A-11C. Similarly, connector (200) can also include an opposing radial cam or second follower member (240) comprised of a projection having a declining ramp that starts at a high top flat region, ledge, or plateau (240A) followed by a first lower sloping region (240B) followed by a second lower sloping region (240C) and further followed by a third lower sloping region (240D), wherein third lower sloping region (240D) is lower in height than high top flat region (240A). Specifically, high top flat region (240A) can be comprised of a flat smooth surface that engages either of ledge or plateau regions (330A) or (340A) of opposing third and fourth cam components (330) or (340), respectively, such as shown in FIGS. 7A-7B and 11A. Similarly, second lower sloping region (240C) can abut, translate, engage, and slide along either of opposing downward sloping regions (330C) or (340C) of opposing third and fourth cam components (330) or (340), respectively, such as shown in FIGS. 7A-7B and 11A-11C.

Still referring to FIGS. 5-6, button (250) of device (100) is adapted to engage connector (200). Here, button (250) can include a third follower member (252) (e.g., a protrusion) having a curved and smooth distal end. In operation, the distal end of third follower member (252) is configured to abut against, translate, and slide along the outer surface of connector (200). In particular, third follower member (252) can engage and slide (or follow) along first and second cam components (210) and (220). Here, first and second cam components (210) and (220) (on opposing sides of connector (200)) can generally be comprised of cut-out areas from the side walls of connector (200) that define a sloping incline and decline ramp. Specifically, first cam member (210) can include a first sloping decline region (210A) that is followed by a second sloping decline region (210B) that is followed by middle region (210C) further followed by a sloping incline region (210D). Similarly, opposing second cam member (220) can include a first sloping decline region (220A) that is followed by a second sloping decline region (220B) that is followed by middle region (220C) further followed by sloping incline region (220D).

FIGS. 7A-7B illustrate an interior region of first housing (300) of device (100). Here, first housing (300) can include therein third and fourth cam components (330) and (340), wherein third and fourth cam components (330) and (340) are each comprised of a sloping ramp arranged in a spiral or helical configuration. Here, third cam member (330) can include a ledge or plateau region (330A) followed by a downward sloping region (330C). Similarly, fourth cam member (340) can include a ledge or plateau region (340A) followed by a downward sloping region (340C). In addition, a partial upstanding cylindrical component (310) (e.g., a ring) with side walls can be disposed between third and fourth cam components (330) and (340). While in one embodiment third and fourth cam components (330) and (340) can include the same downward slope, it is contemplated within the scope of the present disclosure described herein that each cam member may have any sloping surface. In operation, third cam member (330) is adapted to engage first follower member (230) and fourth cam member (340) is adapted to engage second follower member (240), wherein each follower member (230) and (240) slides and translates along the surfaces of third and fourth cam members (330) and (340), respectively. It is contemplated within the scope of the present disclosure described herein that third and fourth cam components or members (330) and (340) may also be referred to herein as follower components or members.

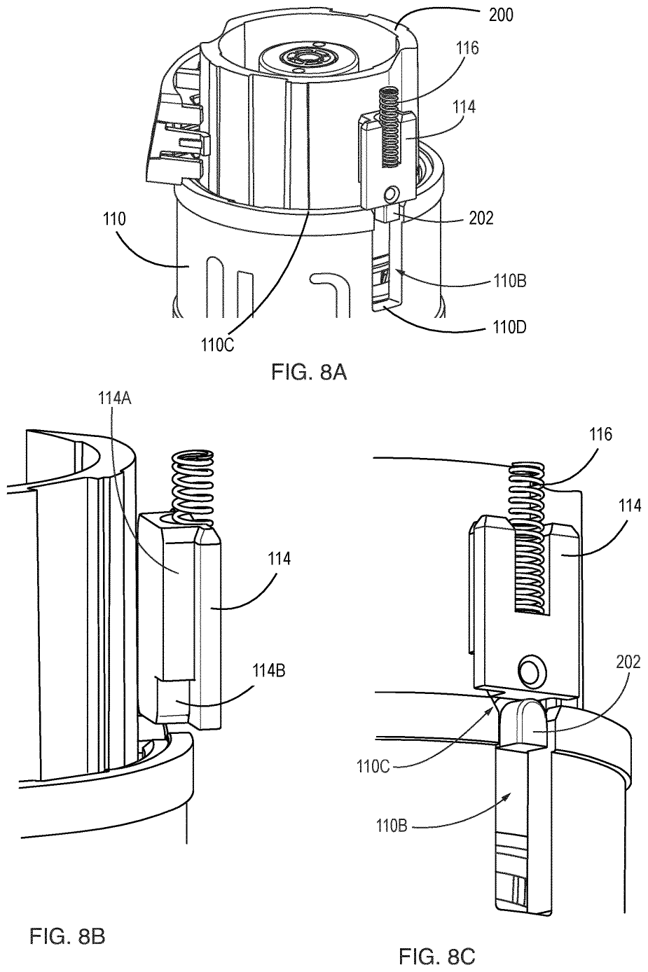

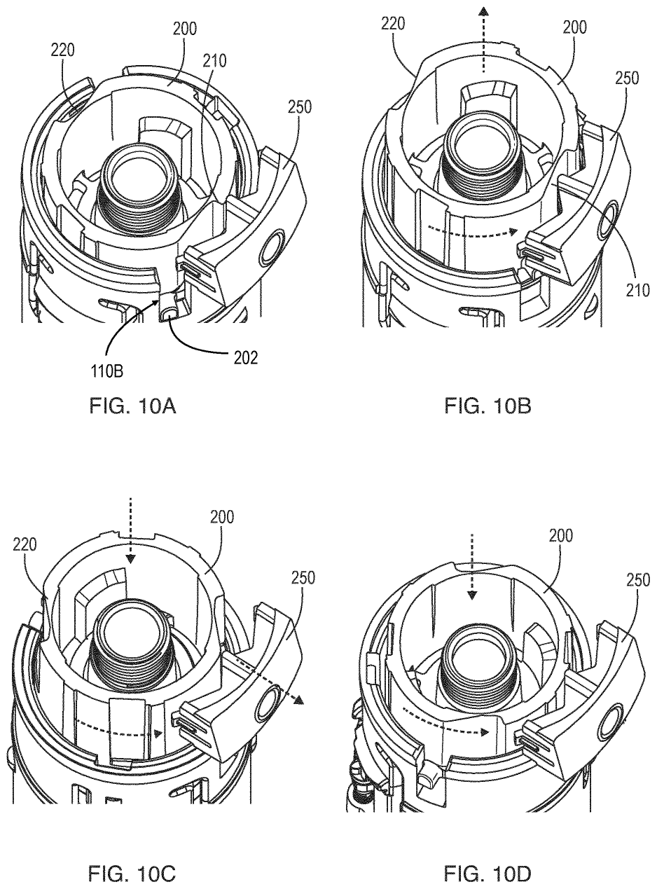

FIGS. 8A-8C illustrate a non-limiting exemplary embodiment of sliding member (114) and sliding spring (116). Here, second housing (110) can include a channel (110B) that allows a protruding tab member (202) of connector (200) to slide (downward or upward) within channel (110B) and further partially rotate within the channel (110B). As shown in FIGS. 8A-8C, connector (200) is shown in its rest position (i.e., liquids/medicament already dispensed). Once device (100) is primed and its actuated position (i.e., prepared for dispensing of liquid/medicament) via rotation of connector (200), then sliding member (114) can slide rotationally along the top lip surface (110C) of second housing (110) until it reaches another channel (110B) (on an opposing side, not shown), wherein the protruding members (114A) and (114B) engage the opposing channel (110B) and slide downward therein. Channel (110B) can also include chamfered top corner regions (110C) to allow for sliding member (114) to more easily slide therein. Here, both opposing channels (110B) include a width that is larger than the width of tab member (202) of connector (200). Specifically, channel (110B) is sized such that it allows tab member (202) to move in a radial direction that is about seven (7) degrees of free movement. In other words, tab member (202) is provided with about seven (7) degrees of rotational movement within the confines of channels (110B). However, it is contemplated within the scope of the present disclosure described herein that tab member (202) may move in any amount such as from about one (1) degree to about thirty (30) degrees of radial movement or along any arc length. In operation, once connector (200) is in its primed position, then tab member (202) can rest on or near bottom surface (110D) of channel (110B) (FIG. 10A), until button (250) is actuated and connector (200) is released upwards to dispense the liquid/medicament.

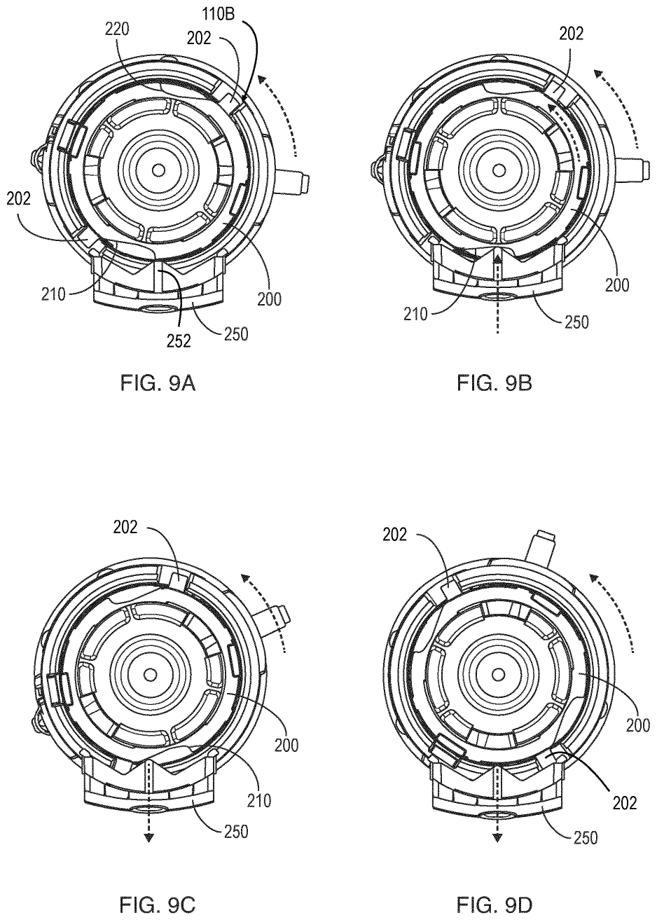

FIGS. 10A-11D illustrate one non-limiting exemplary embodiment of a method of operation of inhaler device (100), namely, a method of arming, priming, or actuating the inhaler device (100) in order to dispense atomized liquid or medicament. FIGS. 9A and 10A illustrate device (100) in its primed/actuated position (i.e., prepared to dispense liquid/medicament). Here, third follower member (252) of button (250) is shown resting on ledge near the opening of first cam member (210) of connector (200). Once a user presses button (250) inward on device (100), then the process will move to FIGS. 9B and 10B, wherein device (100) releases and dispenses atomized liquid or medicament from a nozzle of device (100) via the mouthpiece component (300A), further via the pumping action of connector (200). At this step, third follower member (252) first engages and slides within surfaces (210A) and (210B) of first cam member (210) (or similarly second cam member (220)). Next, at FIGS. 9B and 10B, connector (200) is shown in its fully released state (i.e., resting position). Here, the user can then prime device (100) by relative rotational movement of the first housing (300) and third housing (102), thereby priming the device for its next liquid dispensing session (or actuated state). Here, third follower member (252) further slides along first or second cam components 210 or 220 along surfaces 210C and 210D or 220C and 220D, thereby translationally pushing button (250) outwards back to its primed position. As shown in FIGS. 9B-9D and 10B-10D, third follower member (252) of button (250) can slide along the surface of first cam member (210) as connector (200) is moving in a counter-clockwise direction along with the body of device (100). As device (100) is further primed, connector (200) is shown reciprocating linearly downward such that it returns to its primed or actuated position, such as shown in FIGS. 9A and 10A.

Still referring to FIGS. 10A-11D, once connector (200) is primed again, then third follower member (252) of button (250) will rest near the opening of the next cam member, namely, second cam member (220), in order to repeat the same prior operation of device (100) dispensing the liquid/medicament via actuation of button (250) again. It is noted that once button (250) is depressed or translated inward, it will initiate connector (200) to rotate in a counterclockwise direction independent of first, second, and third housings (300), (110), and (102) and further reciprocate or articulate in an upwards or longitudinal linear direction via spring (106). Here, the travel distance of connector (200) is limited to about seven (7) degrees of rotational movement via tabs (202) within the confines of channels (110B), as previously discussed herein, thereby allowing the device to dispense a pre-defined single dose of the liquid/medication via its pumping unit or pumping chamber.

FIGS. 11A-11D illustrate a non-limiting exemplary embodiment of first follower member (230) of connector (200) following third cam member (330) of first housing (300), wherein second follower member (240) and fourth cam member (340) will include the same or similar operation. As shown in FIG. 11A, while device (100) is in its primed configuration, region (230A) of first follower member (230) can rest or be held on the surface of ledge or plateau (340A). Once device (100) is actuated via the depressing of button (250), then the process can move to the configuration shown in illustration of FIG. 11B. At FIG. 11B, first follower member (230) is shown sliding upwards along the walls of ring (310), such that the surface region (230C) of first follower member (230) engages the surface region (330C) of third cam member (330). In particular, surfaces (230C) and (330C) each have substantially the same slope and angle such that each of the respective surfaces are fully engaged and in contact with each other as they slide and translate relative to each other. Next, first follower member (230) slides and moves rotationally downward along sloping surface of third cam member (330) until it reaches ledge (330A) of third cam member (330) where it is held or secured in its primed position, wherein the same prior operation repeats with respect to fourth cam member (340) of first housing (300) upon actuation of button (250).

It is contemplated within the scope of the present disclosure described herein that any of the aforementioned features, components, and members of device (100) can be made of any material, including but not limited to plastics, polymers, acrylic, polycarbonate, polyethylene, polypropylene, polyvinyl chloride, acrylonitrile-butadiene-styrene, metal, aluminum, or any other suitable material.

From the foregoing it will be seen that the present disclosure described herein is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts described herein, except insofar as such limitations are included in the following claims. Further, it will be understood that certain features and sub-combinations are of utility and may be employed without reference to other features and sub-combinations. This is contemplated by and is within the scope of the claims.

### LIST OF REFERENCES

100 Inhaler device
102 Third housing or lower body
104 Support member
106 Biasing member (e.g., spring)
110 Second housing or intermediate body
110B Channel of second housing (110)
110C Top lip surface of second housing (110)
110D Bottom surface of channel (110B)
112 Riser pipe and holder
114 Sliding member
114A Protruding member of sliding member (114)
114B Protruding member of sliding member (114)
116 Slider spring
200 Connecting member
200A Piping
202 Tab member of connecting member (200)
202A Protrusions, prongs, or tabs of connecting member (200)
204 Cut-out or ledge of connecting member (200)
210 First cam member
210A First sloping decline region of first cam member (210)
210B Second sloping decline region of first cam member (210)
210C Middle region of first cam member (210)
210D Sloping incline region of first cam member (210)
220 Second cam member
220A First sloping decline region of second cam member (220)
220B Second sloping decline region of second cam member (220)
220C Middle region of second cam member (220)
220D Sloping incline region of second cam member (220)
230 First follower member
230A High top flat region, ledge, or plateau of first follower member (230)
230B First lower sloping region of first follower member (230)
230C Second lower sloping region of first follower member (230)
230D Third lower sloping region of first follower member (230)
240 Second follower member
240A High top flat region, ledge, or plateau of follower member (240)
240B First lower sloping region of follower member (240)
240C Second lower sloping region of follower member (240)
240D Third lower sloping region of follower member (240)
250 Button
252 Third follower member (e.g., protrusion or protruding member) of button (250)
300 First housing or upper body
300A Mouthpiece
310 Cylindrical component or ring
330 Third cam member
330A Ledge or plateau region of third cam member (330)
330C Downward sloping region of third cam member (330)
340 Fourth cam member
340A Ledge or plateau region of fourth cam member (340)
340C Downward sloping region of fourth cam member (340)
350 Cap
352 Cover or tab member
354 Hinge
356 Locking and release member button

1. An inhalation device (100), comprising:
a first housing (300);
a connecting member (200) at least partially disposed within the first housing (300) of the inhalation device (100), wherein the connecting member (200) comprises a first cam member (210) and a second cam member (220) on its exterior region and a first follower member (230) and a second follower member (240) in its interior region;
a button (250) secured to the first housing (300) and further engaged with the first cam member (210) or the second cam member (220) of the connecting member (200); and
a biasing member (106) secured to one end of the connecting member (200), such that the connecting member (200) is adapted to at least partially reciprocate within the first housing (300) in order for the inhalation device (100) to dispense a liquid.

2. The inhalation device (100) of claim 1, wherein the first housing (300) further comprises:
a third cam member (330) and a fourth cam member (340), wherein the third cam member (330) is adapted to engage the first follower member (230) and the fourth cam member (340) is adapted to engage the second follower member (240).

3. The inhalation device (100) of any of the preceding claims, wherein the button (250) further comprises:
a third follower member (252), wherein the third follower member (252) is adapted to engage either the first cam member (210) or the second cam member (220).

4. The inhalation device (100) of any of the preceding claims, wherein:
the inhalation device (100) further comprises a second housing (110) comprising a channel (110B);
the connecting member (200) further comprises a tab member (202); and
the tab member (202) is adapted to at least partially rotate within the channel (110B).

5. The inhalation device (100) of claim 4, wherein the tab member (202) is adapted to rotate up to about 7-degrees within the channel (110B) of the second housing (110).

6. The inhalation device (100) of claim 4 or claim 5, further comprising:
a sliding member (114), wherein the sliding member (114) is adapted to slide within the channel (110B) of the second housing (110).

7. The inhalation device (100) of any one of claims 2 to 6, wherein the third cam member (330) and fourth cam member (340) are each in a helical or spiral configuration.

8. The inhalation device (100) of any of the preceding claims, wherein the first follower member (230) and the second follower member (240) each comprise a flat surface region followed by a sloped surface region.

9. The inhalation device (100) of any of the preceding claims, wherein the first cam member (210) and the second cam member (220) are each disposed at about 180-degrees relative to each other.

10. The inhalation device (100) of any of the preceding claims, wherein the first follower member (230) and the second follower member (240) are each disposed at about 180-degrees relative to each other.

11. The inhalation device (100) of any of the preceding claims, further comprising a riser pipe (112) at least partially disposed through a center of the connecting member (200).

12. The inhalation device (100) of claim 11, wherein the connecting member (200) is adapted to reciprocate relative to the riser pipe (112).

13. The inhalation device (100) of claim 11 or 12, wherein the riser pipe (112) is fixed relative to the first housing (300), and the connector (200) reciprocates and moves as a pumping unit relative to the riser pipe (112) for dispensing the liquid.

14. The inhalation device (100) of any of the preceding claims, wherein the biasing member (106) is a compression spring secured to one end of the connecting member (200) .

15. The inhalation device (100) of any one of claims 2 to 14, wherein the first (210), second (220), third (330), or fourth (340) cam members are each comprised of sloping surfaces.
